(19) United States
(12) Patent Application Publication
(10) Pub. No.: US 2026/0191877 A1
(43) Pub. Date: Jul. 9, 2026

(54) APPLICATION OF LONG-ACTING ß-LACTAM ANTIBIOTICS IN THE PREPARATION OF DRUGS FOR THE PREVENTION OR TREATMENT OF PERIOPERATIVE NEUROCOGNITIVE IMPAIRMENT

(71) Applicant: Fujian Medical University Union Hospital, Fuzhou (CN)

(72) Inventor: Zhongmeng Lai, Fuzhou (CN)

(21) Appl. No.: 19/556,172

(22) Filed: Mar. 4, 2026

(51) Int. Cl. A61K 31/546; A61P 25/28

(52) U.S. Cl. CPC A61K 31/546; A61P 25/28

(57) ABSTRACT

This invention belongs to the field of pharmaceutical technology and discloses the use of long-acting β-lactam antibiotics in the preparation of drugs for the prevention or treatment of perioperative neurocognitive impairment (PND). The invention represents a ‘repurposed drug’ approach, with ceftriaxone demonstrating prominent advantages such as broad clinical application, established safety profiles, and mature dosing regimens. Its mechanism of action involves inhibiting the C3/C3aR signaling pathway, elevating circulating platelet factor 4 (PF4) levels, thereby reducing neuroinflammation, protecting hippocampal neurons, and ultimately improving cognitive function.

### TECHNICAL FIELD

The present invention belongs to the field of pharmaceutical technology, specifically involving novel applications of long-acting β-lactam antibiotics in the preparation of drugs for the prevention or treatment of perioperative neurocognitive impairment.

### BACKGROUND

Perioperative neurocognitive disorders (PND) are common severe complications in elderly surgical patients. Their clinical manifestations can be classified into postoperative delirium (POD), delayed neurocognitive recovery (dNCR), and postoperative neurocognitive disorder (PNCD) based on the timing of cognitive impairment. POD and dNCR occur within 30 days postoperatively and affect up to 40% of patients aged 60 years or older. When cognitive impairment persists from 31 days to 12 months postoperatively, it is categorized as PNCD. With the continuous increase in surgical procedures worldwide (approximately 310 million annually), the number of elderly patients affected by PND is substantial, leading to prolonged hospital stays, increased medical costs, heightened dependency, and elevated mortality risks, making it a serious public health issue. Currently, there are no FDA-approved effective drugs for the prevention or treatment of age-related PND in clinical practice.

The exact pathogenesis of postoperative neurodegeneration (PND) remains incompletely elucidated, but studies have demonstrated its close association with neuroinflammation. The gut microbiota interacts with the central nervous system (CNS) through the “gut-brain axis.” Dysbiosis of the gut microbiota induced by surgical trauma can activate microglia and astrocytes within the CNS, triggering neuroinflammation and subsequently leading to cognitive impairment. Activation of the complement system, particularly the complement component 3 (C3) and its receptor C3aR signaling pathway, has been confirmed as a key promoter of neuroinflammation. In aged mouse models, surgery upregulates the expression of C3 and C3aR in the brain; meanwhile, elevated C3 levels have also been observed in the cerebrospinal fluid (CSF) of elderly patients with postoperative neurodegeneration (PNCD). Although studies have shown that blocking C3aR in young mouse models improves postoperative cognition, the specific mechanisms of this pathway in age-related PND, particularly its connections with downstream effector molecules, remain unclear.

On the other hand, platelet dysfunction has also been implicated in cognitive aging. Platelet Factor 4 (PF4), also known as CXCL4, is a cytokine released by platelets. Studies have demonstrated that PF4 has the potential to inhibit neuroinflammation and restore aging brain function, with both platelet counts and circulating PF4 levels declining with age. The cognitive benefits of exercise are partially attributed to its ability to promote PF4 release. However, the role of PF4 in the development of age-related PND, as well as whether its level changes are associated with the aforementioned complement system activation, have not been reported.

### SUMMARY

To this end, there is a need to provide long-acting β-lactam antibiotics, particularly ceftriaxone, with novel applications in the development of prophylactic drugs for PND.

To achieve the above objectives, the present invention provides a method for the preparation of long-acting β-lactam antibiotics for the prevention or treatment of perioperative neurocognitive impairment.

Preferably, the long-acting β-lactam antibiotic is ceftriaxone or its pharmaceutically acceptable salt.

Preferably, the perioperative neurocognitive disorder (PND) includes one or more of postoperative delirium (POD), delayed neurocognitive recovery (dNCR), and postoperative neurocognitive disorder (PNCD).

Preferably, the drug is used to block or inhibit the C3/C3aR signaling pathway.

Preferably, the drug prevents or treats perioperative neurocognitive impairment by increasing the level of platelet factor 4 (PF4) in the subject.

Preferably, the drug prevents or treats perioperative neurocognitive impairment by inhibiting neuroinflammation.

Preferably, the pharmaceutical composition comprises a therapeutically effective amount of a long-acting β-lactam antibiotic and one or more pharmaceutically acceptable carriers, diluents, or excipients.

The technical solution described above offers the following advantages:

The technical solution provided by this invention represents a significant advancement over existing technologies. Its core benefit lies in the rigorous clinical research demonstrating that long-acting β-lactam antibiotics (particularly ceftriaxone) can effectively prevent perioperative neurocognitive impairment (PND) in elderly patients during the perioperative period. Compared to the short-acting antibiotic cefazolin, ceftriaxone exhibits unique long-term protective advantages.

Long-acting β-lactam antibiotics serve as prophylactic or therapeutic agents for perioperative neurocognitive dysfunction (PND). In clinical application, these antibiotics counteract surgical-induced thrombocytopenia and decreased circulating platelet factor 4 (PF4) levels by inhibiting the overactivation of the complement C3/C3aR signaling pathway, thereby effectively reducing neuroinflammation and ultimately improving cognitive function.

This invention represents a “repurposed drug” application. Ceftriaxone, the active ingredient, boasts well-established clinical applications, proven safety profiles, and mature dosing regimens. It eliminates the need for lengthy drug development cycles, enabling rapid translation to benefit a broad patient population. The drug offers significant advantages including low cost, rapid onset of action, and easy scalability. In summary, this invention has achieved remarkable breakthroughs in efficacy, novel mechanisms, clinical feasibility, and socio-economic benefits. It has opened new avenues for the prevention and treatment of PND (Postnatal Depression), yielding multifaceted positive outcomes.

### BRIEF DESCRIPTION OF THE DRAWINGS

FIG. 1 shows the effect of ceftriaxone on serum C3 levels in clinical trials.

FIG. 2 shows the effect of ceftriaxone on individual serum PF4 levels in clinical trials.

FIG. 3 shows the effect of ceftriaxone on individual serum platelet counts in clinical trials.

FIG. 4 shows the effect of CR2-crry inhibiting C3 on serum PF4 levels in individual animals during the animal experiment.

FIG. 5 shows the platelet count levels in different aged PND mouse models from Examples 3 and 4 in the animal experiment.

### DESCRIPTION OF EMBODIMENTS

A long-acting β-lactam antibiotic for the preparation of drugs for the prevention or treatment of perioperative neurocognitive impairment.

In some embodiments, the long-acting β-lactam antibiotic is ceftriaxone or its pharmaceutically acceptable salt.

In some embodiments, the perioperative neurocognitive disorder (PND) includes one or more of postoperative delirium (POD), delayed neurocognitive recovery (dNCR), and postoperative neurocognitive disorder (PNCD).

In some embodiments, the subject of the drug is an elderly subject.

In some embodiments, the elderly subject is aged 60 years or older.

In some embodiments, the administration of the drug is performed perioperatively.

In some embodiments, the perioperative administration includes administration during a period prior to, during, and/or after the surgery.

In some embodiments, the pharmaceutical formulation of the drug is an injectable.

In some embodiments, the drug is used to block or inhibit the C3/C3aR signaling pathway.

In some embodiments, the drug prevents or treats perioperative neurocognitive impairment by increasing the level of platelet factor 4 (PF4) in the subject.

In some embodiments, the drug prevents or treats perioperative neurocognitive impairment by inhibiting neuroinflammation.

In some embodiments, the pharmaceutical composition comprises a therapeutically effective amount of a long-acting β-lactam antibiotic and one or more pharmaceutically acceptable carriers, diluents, or excipients.

To elaborate the technical content, structural features, objectives and effects of the technical solution, the following section provides a detailed explanation with specific implementation examples.

### Example 1

Application of ceftriaxone in prevention of perioperative neurocognitive disorder in elderly patients

### 1.1 Study Design and Patients

This study was a randomized, double-blind, prospective clinical trial. A total of 40 patients aged ≥60 years who planned to undergo radical maxillofacial surgery for oral malignant tumors under general anesthesia were enrolled. Exclusion criteria included severe brain lesions, psychiatric disorders, cognitive impairment, history of stroke, hepatic or renal insufficiency, and hypersensitivity to the investigational drug.

This study enrolled a total of 40 elderly patients (>60 years old) who underwent maxillofacial neck resection. Two patients were excluded for non-compliance with the inclusion criteria. Neurocognitive function was assessed preoperatively and at 7, 30, and 180 days postoperatively using the Mini-Mental State Examination (MMSE) and Mini-Cognitive Assessment (MoCA). Initially, 38 participants were randomly assigned to receive cefazolin or ceftriaxone treatment. Three patients were excluded due to postoperative complications and loss to follow-up. Ultimately, 35 subjects were included in the analysis, divided into the cefazolin group (n=18) and the ceftriaxone group (n=17). The study protocol was approved by the hospital ethics committee, and all patients provided informed consent.

### 1.2 Administration Scheme

Ceftriaxone group: Intravenous administration of ceftriaxone (1 g) preoperatively. If the operative time exceeds 3 hours or the blood loss exceeds 1500 mL, an additional dose is administered intraoperatively; another dose is added after 6 hours. Postoperatively, ceftriaxone (2 g) is administered intravenously daily in combination with metronidazole (500 mg). Metronidazole is discontinued on the 5th postoperative day, and ceftriaxone is discontinued on the 7th postoperative day.
Cefazolin group: The same dosing regimen and dosage as the ceftriaxone group were adopted, with ceftriaxone replaced by cefazolin.

### 1.3 Efficacy Evaluation

Neurocognitive function was assessed using the Chinese version of the Mini-Mental State Examination (MMSE) and Montreal Cognitive Assessment (MoCA) by unblinded evaluators at preoperative and postoperative time points at 1 week, 1 month, and 6 months. A score <23 was considered indicative of cognitive impairment.

The MMSE includes orientation (10 points), calculation (5 points), memory (3 points), delayed recall (3 points), and language (9 points), with a total score of 30 points. The MoCA assesses naming ability (3 points), orientation (6 points), delayed recall (5 points), abstract thinking (2 points), language (3 points), visuospatial ability (5 points), and attention (6 points), also with a total score of 30 points.

### 1.4 Results

| | Cefazolin group | | | Ceftriaxone group | | | | | |
|---|---|---|---|---|---|---|---|---|---|
| | | Number | | | Number | | | | |
| | | of mild | 95% | | of mild | 95% | | 95% | |
| Time | Number | PNCD | confidence | Number | PNCD | confidence | | confidence | |
| point | of | cases | interval | of | cases | interval | odds | interval | |
| (days) | cases | (%) | (%) | cases | (%) | (%) | ratio | (%) | P price |
| 7 ( | 18 | 3 (16.7%) | 0-33.7 | 17 | 6 (35.3%) | 12.8-57.8 | 2.73 | 0.59-11.35 | 0.264 |
| 30 | 18 | 12 (66.7%) | 45.6-87.8 | 17 | 2 (17.6%) | 0-35.4 | 0.07 | 0.01-0.42 | 0.0016** |
| 180 | 18 | 13 (72.2%) | 52-92.4\| | 17 | 5 (29.4%) | 7.6-52.2 | 0.16 | 0.04-0.71 | 0.0184* |

Note:
*p < 0.05,
**p < 0.01;
p-values calculated using Fisher's exact test

The results showed that at 1 week postoperatively, there was no significant difference in the incidence of cognitive impairment between the cefazolin group (3/18, 16.7%) and the ceftriaxone group (6/17, 35.3%) (odds ratio [OR] 2.73 [95% confidence interval (CI), 0.59-11.35]; p=0.264). However, at 1 month postoperatively, there was a significant difference in the incidence of mild postoperative noncognitive disorder (PNCD) between the cefazolin group (12/18 [66.7%]) and the ceftriaxone group (2/17 [17.6%]) (OR 0.07 [95% CI, 0.01-0.42]; p=0.0016). Additionally, this difference persisted at 6 months postoperatively (cefazolin group 13/18 [72.2%] vs ceftriaxone group 5/17 [29.4%]; OR 0.16 [95% CI, 0.04-0.71]; p==0.0184) (Table 1). These findings indicate that perioperative administration of ceftriaxone can effectively reduce the long-term incidence of PNCD in elderly patients.

### Example 2: Effect of Ceftriaxone on Serum Biomarker Levels in Patients

### 2.1 Sample Collection and Testing

Blood samples were collected on the day before surgery and on the 1st, 2nd, and 5th postoperative days in the patient as described in Example 1. The levels of serum complement C3 and platelet factor 4 (PF4) were detected using the enzyme-linked immunosorbent assay (ELISA).

### 2.2 Results

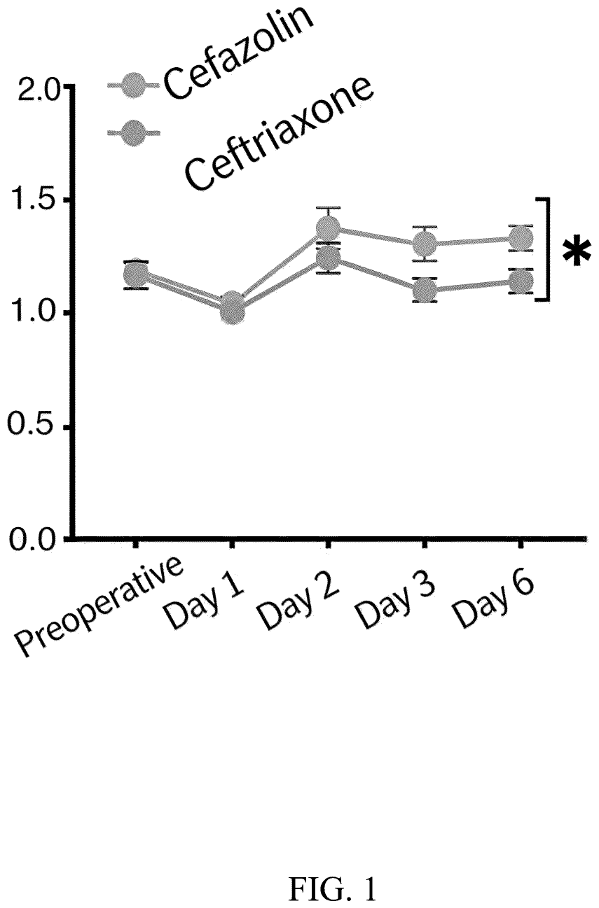

Serum complement C3 and PF4 levels were measured preoperatively and on days 1, 2, 3, and 6 postoperatively. Both groups exhibited elevated serum C3 levels, with the cefazolin group showing a higher incidence of mild PNCD and higher C3 levels compared to the ceftriaxone group (FIG. 1, vertical axis representing serum C3 levels, mg/ml).

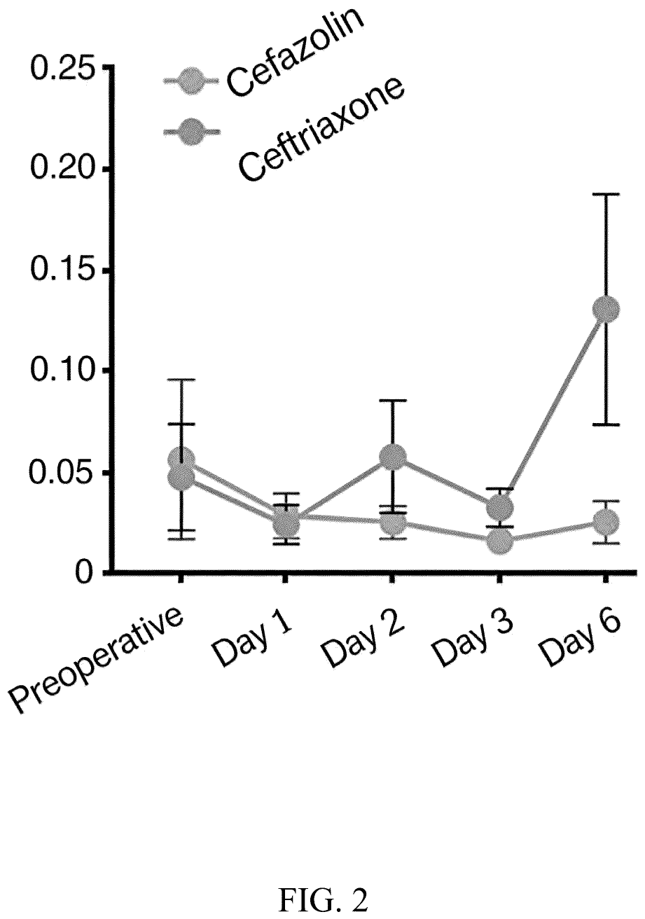

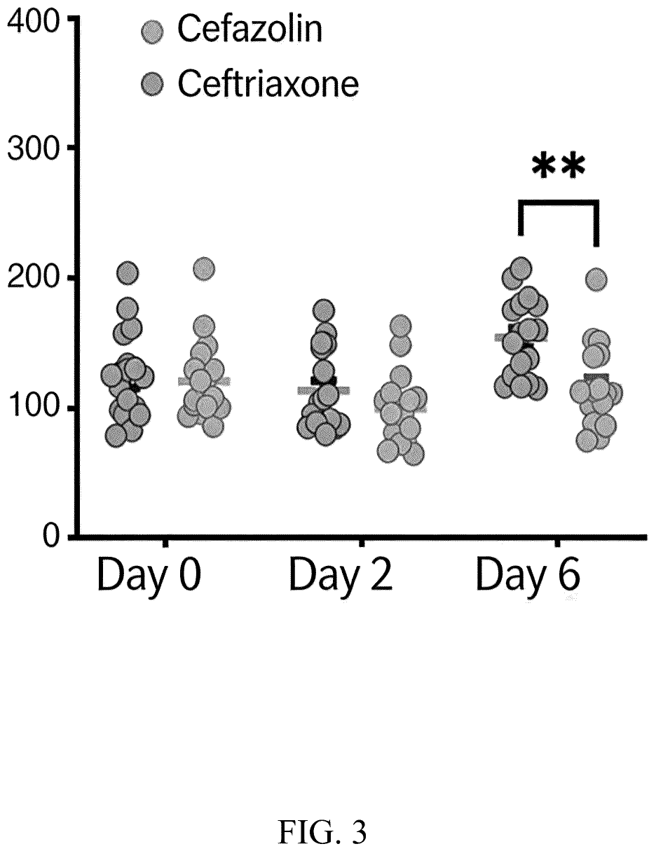

Serum PF4 levels and platelet counts, known to counteract age-related neuroinflammation, were significantly higher in the ceftriaxone group (FIG. 2: vertical axis represents serum PF4 levels, mg/ml; FIG. 3: platelet count, 10\hat{ }9/L). These observations indicate that elevated serum C3 levels are significantly associated with an increased incidence of mild PNCD, while elevated PF4 levels may provide a protective effect against mild PNCD in elderly patients.

### Example 3: Therapeutic Effects of Inhibiting the C3/C3aR Signaling Pathway in an Elderly PND Mouse Model

### 3.1 Animal Models and Administration

Surgical group: 19-20 month old male wild-type (WT) C57BL/6J mice were used to establish the PND model via left common carotid artery exposure surgery. Thirty minutes prior to surgery, the experimental group mice were intravenously administered with the C3 selective inhibitor CR2-crry (0.25 mg).

Control group: No surgical procedures were performed.

Positive control group: C3 gene knockout (C3−/−) mice were used.

### 3.2 Behavioral and Pathological Assessment

Cognitive function was assessed using open-field tests, novel object recognition tests, and fear conditioning tests conducted 3-7 days postoperatively. The results demonstrated that compared to solvent-treated wild-type surgical mice, CR2-crry-treated wild-type surgical mice and C3−/−surgical mice exhibited significantly improved performance in novel object recognition and fear memory tests, indicating reduced cognitive impairment.

This study demonstrates that pharmacological inhibition of the C3 signaling pathway effectively alleviates neuroinflammation and neuronal damage in aged PND models, thereby improving cognitive function.

### Example 4: Therapeutic Effects of Platelet Factor 4 (PF4) Supplementation in an Elderly PND Mouse Model

### 4.1 Animal Models and Administration

Senile wild-type (WT) C57BL/6J mice aged 19-20 months were used. Seven days prior to carotid artery surgery, the experimental group mice received intravenous injection of recombinant PF4 peptide (500 ng) daily, while the control group received an equivalent volume of saline as a solvent.

### 4.2 Behavioral and Pathological Assessment

The behavioral tests were conducted as described in Example 3. The results demonstrated that, compared to the solvent group, the PF4-treated mice exhibited significantly prolonged exploration time toward novel objects in the novel object recognition test and significantly prolonged freezing time in the fear conditioning reflex test, indicating preserved cognitive function.

This implementation demonstrates that direct supplementation of PF4 can mimic the protective effects of ceftriaxone and C3 inhibitors by mitigating neuroinflammation and preserving neuronal architecture to counteract PND.

### Example 5: Regulatory Effects of the C3/C3aR Signaling Pathway on Platelets and PF4

### 5.1 Sample Testing

In the aged PND mouse models described in Examples 3 and 4, blood and serum samples were collected on the 1st postoperative day. Platelet counts were performed using an automated hematology analyzer, and serum PF4 levels were detected with an ELISA kit.

### 5.2 Results

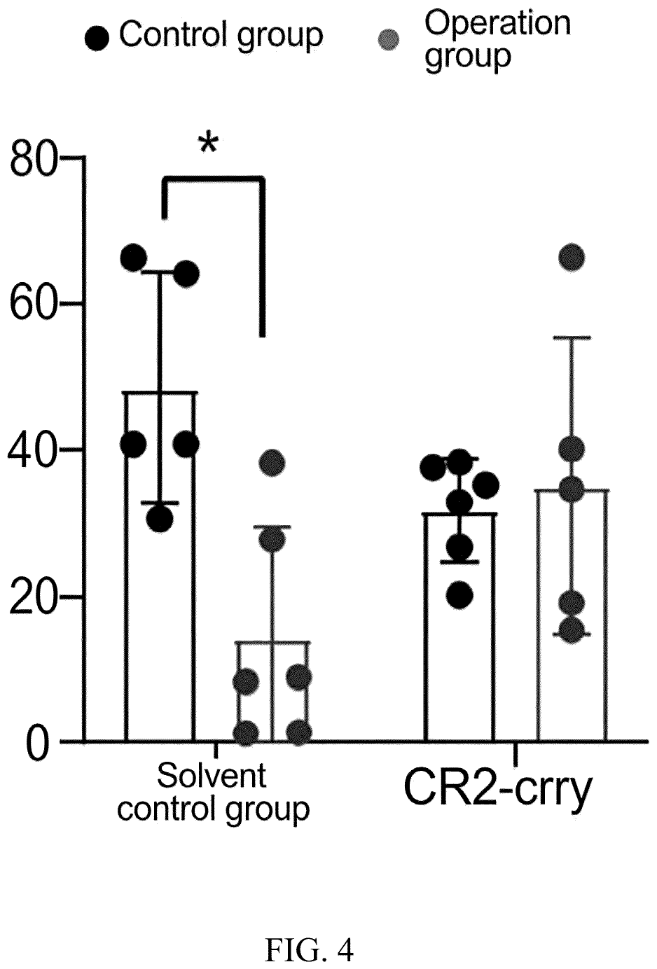

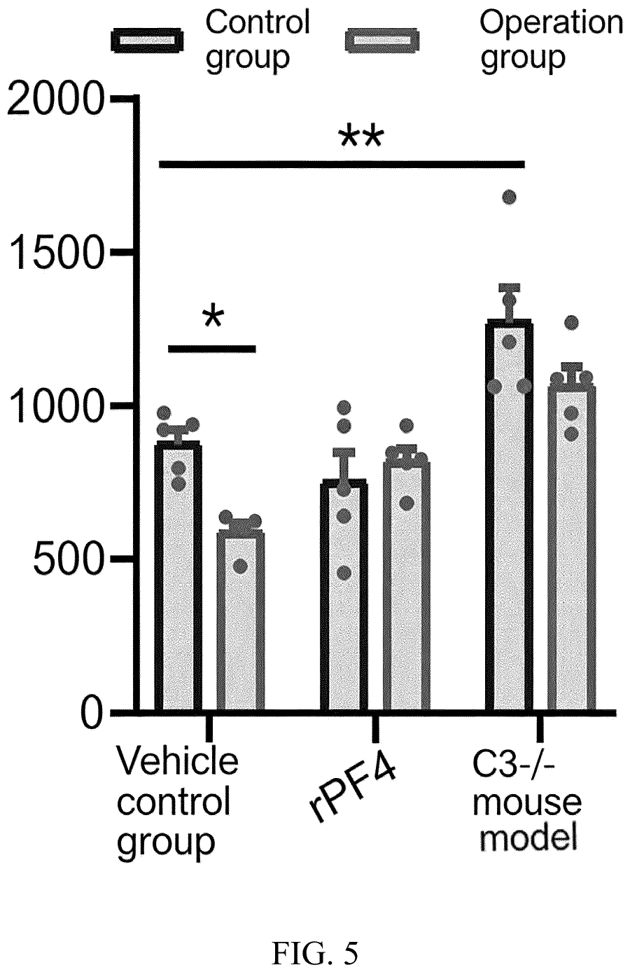

Postoperative analysis revealed a significant reduction in platelet count and serum PF4 levels in aged wild-type mice (FIG. 4: serum PF4 level, μg/ml; FIG. 5: platelet count, 10\hat{ }9/L). In contrast, C3−/−mice maintained elevated platelet counts both pre- and post-surgery, with the postoperative decline in serum PF4 being markedly suppressed. Similarly, CR2-crry-mediated inhibition of C3 in wild-type mice effectively prevented the decrease in serum PF4 levels. This study directly demonstrates that activation of the C3/C3aR signaling pathway serves as the upstream critical event underlying surgery-induced platelet depletion and PF4 downregulation, elucidating the core mechanism of this invention.

In conclusion, the aforementioned embodiments fully demonstrate that the long-acting β-lactam antibiotics (represented by ceftriaxone) described in this invention exhibit significant efficacy in preventing and treating perioperative neurocognitive impairment. This effect is achieved through a novel mechanism involving the inhibition of the C3/C3aR signaling pathway and the elevation of PF4 levels.

It should be noted that in this document, relational terms such as “first” and “second” are used solely to distinguish one entity or operation from another, without implying any actual relationship or sequence between them. Furthermore, terms like “include,” “contain,” or their variants are intended to cover non-exclusive inclusion, meaning that processes, methods, items, or terminal devices comprising a series of elements not only include those explicitly listed but also other elements not explicitly listed, or elements inherent to such processes, methods, items, or terminal devices. Without further limitation, elements defined by statements such as “include . . . ” or “contain . . . ” do not exclude the presence of additional elements in the processes, methods, items, or terminal devices. Additionally, in this document, terms such as “greater than,” “less than,” and “exceed” are understood to exclude the base number, while “above,” “below,” and “within” are understood to include the base number.

Although the aforementioned embodiments have been described, those skilled in the art may make additional modifications to these embodiments upon understanding the fundamental inventive concept. Thus, the above description constitutes only embodiments of the present invention and does not limit its patent protection scope. Any equivalent structural or process modifications derived from the disclosure of this invention, or any direct or indirect application to other related technical fields, shall likewise be included within the scope of the patent protection of the present invention.

What is claimed is:

1. Use of long-acting β-lactam antibiotics in the preparation of drugs for the prevention or treatment of perioperative neurocognitive impairment.

2. The use according to claim 1, characterized in that the long-acting β-lactam antibiotic is ceftriaxone or its pharmaceutically acceptable salt.

3. The use according to claim 1, characterized in that the perioperative neurocognitive impairment comprises one or more of postoperative delirium, delayed neurocognitive recovery, and postoperative neurocognitive impairment.

4. The use according to claim 1, characterized in that the drug is used to block or inhibit the C3/C3aR signaling pathway.

5. The use according to claim 1, characterized in that the drug prevents or treats perioperative neurocognitive impairment by increasing the level of platelet factor 4 in the subject.

6. The use according to claim 1, characterized in that the drug prevents or treats perioperative neurocognitive impairment by inhibiting neuroinflammation.

7. The use according to claim 6, characterized in that the pharmaceutical composition comprises a therapeutically effective amount of a long-acting β-lactam antibiotic and one or more pharmaceutically acceptable carriers, diluents, or excipients.
